(19) United States
(12) Patent Application Publication
(10) Pub. No.: US 2004/0014944 A1
(43) Pub. Date: Jan. 22, 2004

(54) Method and system useful for structural classification of unknown polypeptides

(76) Inventors: Michal Linial, Jerusalem (IL); Elon Portugaly, Jerusalem (IL)

Correspondence Address: Anthony Castorina, G E Ehrlich, Suite 207, 2001 Jefferson Davis Highway, Arlington, VA, 22202, US

(21) Appl. No.: 10/399,751

(22) PCT Filed: Oct. 24, 2001
(86) PCT No.: PCT/IL01/00980

(51) Int. Cl. G06F 19/00; G01N 33/48; G01N 33/50; C07K 14/00

(52) U.S. Cl. 530/350; 702/19

(57) ABSTRACT

A method of identifying a polypeptide candidate most likely to have undefined structural/functional elements is provided. The method is effected by (a) classifying each cluster of a database of non-structurally clustered polypeptide clusters as: (i) a first cluster, if it includes at least one structurally defined polypeptide; or (ii) a second cluster, if it is devoid of structurally defined polypeptides; (b) determining relational distances between at least one first cluster and a plurality of distinct second clusters according to at least one criteria; and (c) identifying second clusters of the plurality of distinct second clusters which exhibit a relational distance from the at least one first cluster which is greater than a predetermined threshold, thereby identifying the polypeptide candidate most likely to have undefined structural/functional elements.

### FIELD AND BACKGROUND OF THE INVENTION

The present invention relates to a method and system useful for structural classification of unknown polypeptides and, more particularly, to a method which utilizes structural and sequence alignment data to determine the relationship between structurally defined and structurally undefined proteins.

The recently fully sequenced human genome, has been found to contain up to 38,000 genes (Venter J. C. et al., Science 2001, 291:1304) encoding up to an order of magnitude more protein species. It is evident that the information contained therein holds tremendous potential for furthering the development of practical applications in all fields involving the life sciences. However, most proteins remain to be characterized with respect to their structure and function and, although the transcription profiles of the genes encoding these proteins are currently being determined, such data can yield only limited information. In order to fully harness the potential of the information contained in the complete human genome sequence, it will be necessary to systematically determine the 3D structure of the proteins encoded therein.

Knowledge of the three-dimensional (3D) structure of proteins is proving to be crucial for understanding and regulating their biological functions and, as such, is playing an increasingly vital role in the advancement of biomedical science and biotechnology. One such outstanding example has been the structure-based development of protease inhibitors employed in the first effective treatment of human immunodeficiency virus infection (Wlodawer A. and Vondrasek J. Annu Rev Biophys Biomol Struct. 1998, 27:249).

Despite recent developments in structural determination methods (Montelione, 1999) it is not currently feasible to experimentally define the three dimensional structure of hundreds of thousands of proteins. Thus, at present, prediction of key structural properties of protein must rely upon available sequence data and structural data derived from structurally defined proteins.

Attempts to computationally determine a protein's structure based on sequence data alone have met with limited success, partly due to the shortage of solved protein structures utilizable as models.

With the absence of accurate computational models, the challenge at present is to define a relatively small set of representative proteins which when structurally defined would enable modeling of novel protein folds (Sali, 1998). Using such model proteins and techniques such as comparative modeling and fold recognition will enable large-scale structural predictions (Koehl, 1999).

It is generally accepted that reasonable predictions are possible if the unknown protein and at least one of the known model templates share at least 30% sequence identity (Sali, 1998). Thus, in order to enable computational resolution of any unknown protein, a diversified set of structurally defined model proteins must be selected such that all possible structural folds are represented.

Data accumulated from genomic sequencing accelerated the development of computational approaches which “superimpose” 3D structures on genomic data. This include improved methods for detecting distal homologues (Huynen, 1998; Teichmann, 1998; Wolf, 1999) and exhaustive 3D model building (Sanchez, 1998; Fischer, 1997; Jones, 1999; Koehl, 1999). In addition, several pilot projects in which known structures serve as models for assigning structure to other related proteins of specific organisms were initiated in recent years.

Although numerous computational models for predicting protein structures exist in the art, such models depend upon available structural data which at present lacks the diversity needed for accurate computational predictions of structurally undefined proteins.

The question of how many targets proteins are needed to be selected in the frame of a structural genomics effort is linked to the estimated number of protein folds that exist in the entire protein space. This number is estimated as ranging from 700 to over 10,000 folds (Orengo, 1994; Zhang, 1998; Finkelstein, 1987; Govindarajan, 1999; Wang, 1998; Chothia, 1992). The total number of currently known protein folds is 473 according to the SCOP 1.39 classification (Murzin, 1995; Hubbard, 1999) and 635 folds (topologies) according to the CATH 1.5 classification (Orengo, 1997). The rate of fold discovery in recent years confirms that while the number of solved structures is exponentially growing, the fraction of new folds is constantly decreasing (based on the yearly deposit of folds by SCOP and on records from the PDB). It is widely accepted that in order to increase the rate of new fold discovery more suitable and diverse protein targets must be selected.

A critical requirement for the selection of suitable targets is a comprehensive clustering of protein sequences and the identification of clusters which represent novel structural folds; a representative protein of a such clusters can then be selected as a potential candidate for structural determination.

There is thus a widely recognized need for and it would be highly advantageous to have a system and method which can be utilized to identify novel protein folds the structural analysis of which would result in novel structural data capable of substantially improving the accuracy of computational approaches for protein structure modeling and prediction.

### SUMMARY OF THE INVENTION

While reducing the present invention to practice the present inventors have uncovered that structural classification of polypeptide clusters generated according to sequence data can be utilized to uncover structurally undefined proteins which can serve as model proteins for computational determination of novel protein structures.

Thus, according to one aspect of the present invention there is provided a method of identifying a polypeptide candidate most likely to have undefined structural/functional elements comprising: (a) classifying each cluster of a database of non-structurally clustered polypeptide clusters as: (i) a first cluster, if it includes at least one structurally defined polypeptide; or (ii) a second cluster, if it is devoid of structurally defined polypeptides; (b) determining relational distances between at least one first cluster and a plurality of distinct second clusters according to at least one criteria; and (c) identifying second clusters of the plurality of distinct second clusters which exhibit a relational distance from the at least one first cluster which is greater than a predetermined threshold, thereby identifying the polypeptide candidate most likely to have undefined structural/functional elements.

According to another aspect of the present invention there is provided a method of determining putative structural/functional characteristics of a polypeptide having an unknown structure or function, the method comprising: (a) classifying each cluster of a database of non-structurally clustered polypeptide clusters as: (i) a first cluster, if it includes at least one structurally defined polypeptide; or (ii) a second cluster, if it is devoid of structurally defined polypeptides; (b) determining relational distances between at least one first cluster and a plurality of distinct second clusters according to at least one criteria; (c) identifying second clusters of the plurality of distinct second clusters which exhibit a relational distance from the at least one first cluster which is less than a predetermined threshold; and (d) determining the putative structural/functional characteristic of at least one polypeptide of the second clusters according to structural/functional characteristics of the at least one structurally defined polypeptide of the at least one first cluster, thereby determining putative structural/functional characteristics of a polypeptide having an unknown structure or function.

According to further features in preferred embodiments of the invention described below, the method further comprising assigning each of the plurality of distinct second clusters a score according to a relational distance thereof from the at least one first cluster and at least one additional criteria selected from the group consisting of number of polypeptides in cluster, predominant host organism of cluster and average size of polypeptides in cluster.

According to still further features in the described preferred embodiments the database of non-structurally clustered polypeptide clusters is generated according to clustering of sequence data.

According to still further features in the described preferred embodiments the relational distances are determined according to sequence homology between polypeptides of the plurality of distinct second clusters and the at least one first cluster.

According to still further features in the described preferred embodiments each of the clusters of the database of non-structurally clustered polypeptide clusters is further classified according to the number of polypeptide constituents contained therein.

According to still further features in the described preferred embodiments the method further comprising generating the database of non-structurally clustered polypeptide clusters according to clustering of sequence data prior to step (a).

According to still further features in the described preferred embodiments the classifying is repeated a predetermined number of times, each time for clusters generated according to a different homology threshold.

According to yet another aspect of the present invention there is provided a system for identifying a polypeptide candidate most likely to have undefined structural/functional elements, the system comprising a processing unit being for executing a software application designed for: (a) classifying each cluster of a database of non-structurally clustered polypeptide clusters as: (i) a first cluster, if it includes at least one structurally defined polypeptide; or (ii) a second cluster, if it is devoid of structurally defined polypeptides; (b) determining relational distances between at least one first cluster and a plurality of distinct second clusters according to at least one criteria; and (c) identifying second clusters of the plurality of distinct second clusters which exhibit a relational distance from the at least one first cluster which is greater than a predetermined threshold, thereby identifying the polypeptide candidate most likely to have undefined structural/functional elements.

According to still another aspect of the present invention there is provided a system for determining putative structural/functional characteristics of a polypeptide having an unknown structure or function, the system comprising a processing unit being for executing a software application designed for: (a) classifying each cluster of a database of non-structurally clustered polypeptide clusters as: (i) a first cluster, if it includes at least one structurally defined polypeptide; or (ii) a second cluster, if it is devoid of structurally defined polypeptides; (b) determining relational distances between at least one first cluster and a plurality of distinct second clusters according to at least one criteria; (c) identifying second clusters of the plurality of distinct second clusters which exhibit a relational distance from the at least one first cluster which is less than a predetermined threshold; and (d) determining the putative structural/functional characteristic of at least one polypeptide of the second clusters according to structural/functional characteristics of the at least one structurally defined polypeptide of the at least one first cluster, thereby determining putative structural/functional characteristics of a polypeptide having an unknown structure or function.

According to still further features in the described preferred embodiments the software application is further designed for assigning each of the plurality of distinct second clusters a score according to a relational distance thereof from the at least one first cluster and at least one additional criteria selected from the group consisting of number of polypeptides in cluster, predominant host organism of cluster and average size of polypeptides in cluster.

According to still further features in the described preferred embodiments the relational distances are determined by the software application according to sequence homology between polypeptides of the plurality of distinct second clusters and the at least one first cluster.

According to still further features in the described preferred embodiments each of the clusters of the database of non-structurally clustered polypeptide clusters is further classified by the software application according to the number of polypeptide constituents contained therein.

According to still further features in the described preferred embodiments the software application is further designed for generating the database of non-structurally clustered polypeptide clusters according to clustering of sequence data.

The present invention successfully addresses the shortcomings of the presently known configurations by providing a system and method with which accurate identification of potentially new protein folds can be effected.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph illustrating the relationship between top-level ProtoMap clusters and SCOP families.

FIG. 2 illustrates a connected-component composed of 30 different clusters. Each circle represents a cluster with its associated number. The black circle represents an occupied cluster. Edges with weight of >0.1 are shown. Cluster 3960 is occupied by PDB-1BO4 (Erratia marescens aminoglycoside 3-N-Acetyltransferase) which belong to the GCN5 sequence group (Neuwald and Landsman, 1997).

FIG. 3 illustrates determination of the vacant-surrounding-volume of cluster A. Left panel—if no other occupied cluster is present in that connected-component, V is undefined. Right panel—illustrates the difference between measuring distances by the number of steps (S) and by the vacant-surrounding-volume (V).

FIG. 4 illustrates distribution of vacant-surrounding-volumes over the clusters. X-axis, vacant-surrounding-volume. Y-axis, number of clusters with this volume, log scale; values of 1 are marked with a small scale on the X-axis. Occupied clusters (black bar) have a vacant-surrounding-volume of 0. Neighbors to occupied clusters (grey bar) have vacant-surrounding-volume of 1. The number of clusters with undefined vacant-surrounding-volume is 9552 (˜24,000 proteins).

FIGS. 5a-b illustrate the distribution of P-values for 200 runs of the SCOP 1.37-SCOP 1.50 test described in Example 1 hereinbelow. Thick line, prediction according to the present method. Dashed line, prediction using results from PSI-BLAST (Altschul et al., 1990). Fenced line, prediction using results from the Smith-Waterman search (smith and Waterman, 1981). Crossed line, prediction using the present method with ties broken using results from PSI-BLAST, see Example 1 for explanation.

FIGS. 6a-b illustrate the P-values for all 6 (base, test) sets, as derived by the method of the present invention (black dot); PSI-BLAST (open diamond); or the method of the present invention with ties broken by PSI-BLAST (open diamond with black dot). P-values were averaged for 200 runs per test. X-axis—size of sample set. Y-axis—P-value. FIG. 6a—non-occupied sample sets. FIG. 6b—non-occupied-neighboring sample sets.

FIG. 7 is a schematic representation of a globin ProtoMap graph surrounding cluster 3 at e=10⁻⁰ (621 proteins). The 15 clusters indicated by the circles account for 845 proteins. Proteins that belong to globin family are within the grey area. The sizes of the circles are correlated with number of proteins (in increasing order 1-5, 5-50, 51-200 and >200 proteins in a cluster). Edges in the graph indicating the proximity between any two clusters as measured by the quality score. Quality score at e=10⁻⁰ ranges from 0.99 to 0.01. Edges with quality >0.1, 0.06-0.1 and 0.01-0.05 are indicated by a thick, thin and dashed lines, respectively. Note that most connections from the globin map to additional graphs (listed in the rectangle and indicated by the pointing arrows) are associated with edges of low quality score.

FIG. 8a illustrates the distribution of the number of occupied SP-chains in each occupied cluster. Percent of occupied clusters with indicated number of occupied SP-chains (empty bar) and percent of occupied SP-chains in each category (grey bar) are shown. The clusters with largest number of occupied SP-chains are indicated along with the ProSite signature. Note that while 59% of the occupied clusters contain only one occupied SP-chains, more than 73% of the occupied SP-chains are in clusters that contain two or more occupied SP-chains per cluster.

FIG. 8b illustrates representative clusters with occupied multi-domains SP-chains. The geometrical symbols represent different folds within each cluster. The numbers indicate the occurrence of a specific fold combination among the occupied SP-chain in that cluster. The number of the domains and the occupied SP-chains are included. In all 5 examples, the fold with the rectangle symbol was selected as the representative fold of that cluster.

FIG. 9 illustrates base distributions of Dold[V] and Dnew[V] for threshold 0.1. Partition of the vacant volumes to classes was performed as detailed in Example 2. Number of clusters counted in the training set in each class is shown.

FIG. 10 illustrates the probability of having a new fold for various vacant volumes as calculated according to Baye's rule. The dashed line indicates the a priori probability to be new (see Example 2 for detail).

FIGS. 11a-b illustrate the proportion of new folds in each bin. FIG. 11a—number of clusters in bin that were assigned any new structure from SCOP 1.39. FIG. 11b—number of clusters in bin that were assigned a new fold from SCOP 1.39. X-axis—predicted probability to be new assigned to bin. Y-axis—proportion of new folds out of new structures that were assigned to bin. A linear trendline with its r-square value is shown.

### DESCRIPTION OF THE PREFERRED EMBODIMENTS

The present invention is of a system and method which can be used to uncover proteins having new fold structures. Specifically, the present invention can be used to identify protein targets suitable for structural analysis, which when resolved can serve as model templates for computational structural analysis of uncharacterized proteins.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The accuracy of computational structural biology in predicting protein structure is currently limited by a limited diversity of resolved protein folds which can serve as templates.

Thus, new protein folds must be resolved in order to increase the accuracy of computational methodology. However, due to the large number of unresolved proteins which exists, it is at present difficult, if not impossible, to predict which proteins, which when resolved, would further diversify existing fold data.

The present invention provides methodology with which suitable protein targets can be selected prior to structural resolution, thus greatly facilitating future structural/functional determination of uncharacterized proteins.

As used herein, the phrase “relational distance” refers to a value which denotes the relatedness of two proteins or protein clusters. The greater the relationship distance the less functionally and/or structurally related the proteins or protein clusters are and vice versa.

The Examples section which follows describes in detail methods of quantization such a “relational distance”, and methods of applying it in order to determine functional/structural characteristics of structurally undefined proteins. Additional terms and phrases utilized herein are explained throughout the Examples section which follows.

According to one aspect of the present invention there is provided a method of identifying a polypeptide candidate most likely to have undefined structural/functional elements or structural folds.

Such a method is preferably effected using a processing unit, such as a personal computer (e.g., PC, Apple Macintosh), a work station or a main frame provided with suitable software applications designed for executing the method steps described below.

The method according to this aspect of the present invention is effected by first (a) classifying each cluster of a database of non-structurally clustered polypeptide clusters as: (i) a first cluster, if it includes at least one structurally defined polypeptide; or (ii) a second cluster, if it is devoid of structurally defined polypeptides.

The database of non-structurally clustered polypeptide clusters is preferably generated by clustering polypeptide sequences according to sequence homology information as well as other information which can include polypeptide size and host organism. Further description of such clustering is provided in the Examples section which follows with respect to the ProtoMap software application which is described in WO 99/39174, published Aug. 5, 1999.

Following classification of the clusters, which can be repeated several times, each time for a different set of clusters generated according to a different clustering criteria (e.g., a different homology threshold), the classified clusters of group (ii) are scored according to a distance from at least one cluster of group (i). This step is preferably performed to exhaustion, mapping all distance relationships between the clusters of group (ii) and cluster (i), and inter-between clusters of group (ii).

The clusters are then listed according to scoring and the top scoring clusters (most distant from cluster (i) or the clusters which score higher than a predetermined threshold are selected, such clusters have the highest probability to include undefined structural/functional elements and thus are suitable candidates for structural analysis.

Thus, by co-integrating structural and sequence information the methodology of the present invention overcomes limitations inherent to prior art methods of protein clustering. Since such methods almost exclusively rely upon sequence homology data, they are limited by the thresholds used for alignment. In sharp contrast, superimposition of structural information onto databases clustered according to sequence homology information enables extraction of additional information relating to cluster identity.

It will be appreciated that the above described methodology is not only limited in application to the identification of novel protein folds. Since the data extracted remaps protein clusters, the present methodology can also be used to establish relationships between previously unrelated proteins, to remap the evolutionary tree and to assign functional and/or structural characteristics to previously uncharacterized proteins.

Thus, the present invention provides a novel approach for identifying yet uncharacterized protein folds.

The discovery of a novel fold contributes to understanding functional details of entire protein families. Based on the number of structurally characterized families it is estimated that currently there is only 15-25% of the number required to obtain structures for all (95%) folds (Wolf et al., 2000). Thus, methodology for discovering currently missing folds and superfamilies is highly desirable (Holm and Sander, 1997; Marti-Renom et al., 2000; Murzin, 1996).

The present invention presents a statistical-computational method which can greatly increase the rate at which new superfamilies and new folds are discovered and thus provides valuable information regarding biological features of proteins.

It should be noted that not all these proteins that are predicted to have new fold will eventually reveal new folds. About 10% of all folds in SCOP are folds with multiple superfamilies. Extreme cases are the TIM barrel, Ferredoxin, Flavodoxin etc. For many of these superfamilies, sharing the same fold is a result of convergent evolution. Consequently, it is expected that some of the proteins selected as targets will turn out to be new superfamilies that belong to already known folds. In addition, within the target list presented, several of the clusters belong to the same local graphs. In such instances, solving one representative in that graph is expected to enable resolution of other clusters in that graph (see Example 2 for further detail).

Results obtained herein indicate for the first time that related cluster lists of ProtoMap hold information pertaining to fold relationships. Although ProtoMap is based solely on sequence information the present methodology has clearly proved that a global approach which considers relationships between all known sequences, can be used to extract information relating to protein structure at the fold level.

The results obtained by the present studies validate the robustness and accuracy of the present methodology. It will be appreciated that information in ProtoMap's related clusters lists, if mined by more sophisticated tools (i.e. improved algorithms and better choice of data features) will probably provide additional information relating to fold identity of yet uncharacterized proteins.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

The nomenclature and analytical methods used herein are thoroughly explained in the literature and are well known to one ordinarily skilled in the art to which they pertain. For further description, see, for example, the document referenced hereinbelow. All the information contained therein is incorporated herein by reference.

### Example 1

Selecting Candidates for Structural Determination from a Graph of Protein Families

Methods and Results

The present study utilized the ProtoMap 2.0 database (http://www.protomap.cs.huji.ac.il) (Yona et al., 1998). ProtoMap is an automatically generated hierarchical classification of all protein sequences in Swissprot (release 36, 72,623 protein sequences). The present study focused on the top (most relaxed) level of classification (level e-0). In this level there are 13,354 clusters, 7,485 of which are singletons (including one protein). Each cluster in ProtoMap has a weighted list of neighboring clusters (smaller weight—higher significance for relatedness) and thus each level of classification forms a weighted graph. Inspecting the top level graph reveals that in most cases, related clusters encode biological meaningful relations (Linial and Yona, 2000; Portugaly and Linial, 2000; Yona et al., 2000). A key feature of ProtoMap is that the elementary unit for ProtoMap clustering and classification is the whole protein sequence rather than protein domains. This infers that a cluster composed of multi-domain proteins may contain more than one structural entity.

Structural data was provided by SCOP 1.50 which is a hierarchical classification of all known structural domains (http://scop.mrc-lmb.cam.ac.uk/scop). SCOP 1.50 contains ˜23,800 records obtained from more than 10,000 PDB records. These records are classified into ˜1300 families, 820 superfamilies and 548 folds. Many PDB and SCOP records do not contain a Swissprot ID field (due to minor differences in the sequence of the structured protein). Therefore, in order to associate SCOP records with Swissprot records, a strict sequence similarity test was performed, allowing each SCOP record to associate with one Swissprot record; as a result, ˜1200 of the families associated with ˜3200 Swissprot proteins which belong to 1162 clusters in the ProtoMap database. These clusters are referred to herein as “occupied”. 653 of the Swissprot proteins associated with more than one SCOP family (heterogeneous multi-domain proteins).

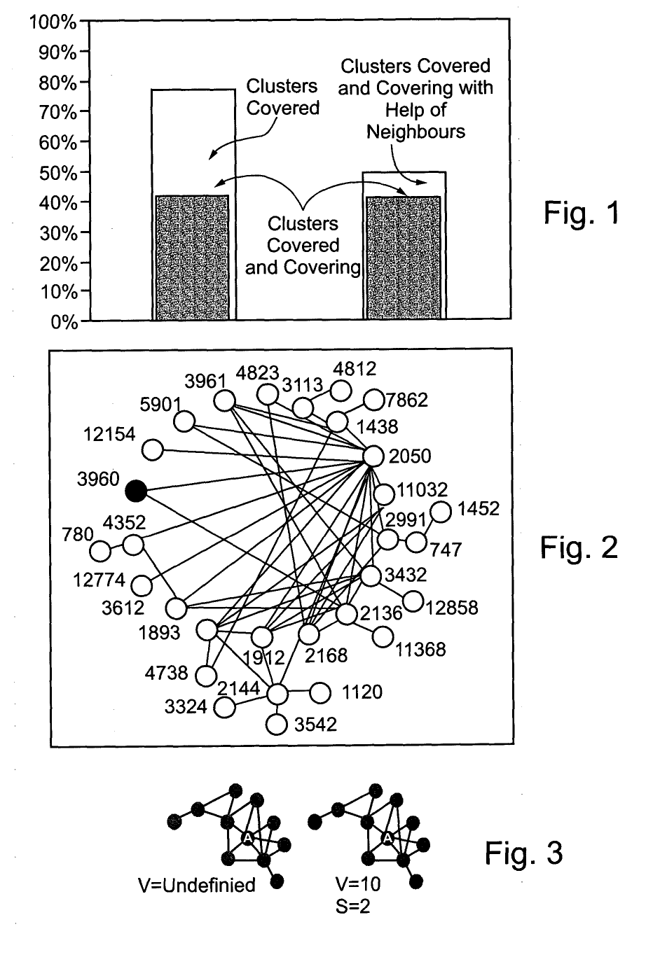

FIG. 1 illustrates the relationship between a top level ProtoMap cluster to a SCOP family. Due to the intrinsic nature of clustering, characteristics of whole proteins and the prevalence of multi-domain proteins a general cluster cannot be expected to correlate with one SCOP family. Occupied clusters that contain more than one solved protein (non-trivial clusters) were examined. A cluster is considered covered by a family if 90% of the solved proteins in this cluster belong to that family. A cluster is considered to be covering a family if 90% of the solved proteins of that family belong to the cluster. A cluster is considered to be covering a family with the help of its neighbors if 90% of the solved proteins of that family belong to the cluster or to one of its neighbors.

The results obtained indicate that 77% of the non-trivial clusters are covered while 41% of the clusters are each covering and being covered by the same family. While a major part of the clusters are correlated with SCOP families, another major part of them are constructed of a portion of a family (77%−41%=36%). In addition, 49% of the clusters are each being covered by a family and cover that family with the help of their neighbors. This contribution of the neighboring clusters is significant mainly in the top three levels of ProtoMap classification, indicating that the edges of the ProtoMap graph include interesting information related to those levels.

Properties of the ProtoMap Graph, with Respect to SCOP Classification

A working hypothesis that distances on ProtoMap graph are consistent with distances between protein structures was examined. Numerous biological tests indeed confirm this hypothesis (Yona et al., 1999). Using this hypothesis as a guideline, it was postulated that if a cluster that is distant in the graph from any a known structure is compared to a cluster that is near and thus more related to a known structure, the former would stand a higher chance of containing a new superfamily or fold.

An example of the biological information included in the graph edges is shown in FIG. 2 which illustrates the complete connected-component of cluster 2050 (For the ProtoMap top level graph, clipped at edge weight of 0.1). Of the 140 proteins examined only cluster 3960 of the connected-component is occupied. The proteins within this connected-component belong to the GCN5 group whose proteins share extremely low sequence similarity. This group consists of proteins with diverged sequences and biological functions from bacteria to man (Neuwald and Landsman, 1997). Almost all proteins were tested and confirmed to belong to that group. The protein structure mapped to cluster 3960 belongs, according to SCOP, to N-acetyltransferase, an NAT family within the CoA-N-acetyltransferase fold. It was further hypothesized that the other proteins in the graph share the same structural identity. Manual inspection using less strict sequence similarity than the one used for mapping uncovered that all other 7 N-acetyltransferase NAT family members have close homologues in this connected component (not shown).

Defining the Vacant Surrounding Volume of a Cluster

To define the vacant surrounding volume of a cluster, the edges of the ProtoMap top level graph were cropped, while retaining edges of weight 0.1 or lower (more significant); the remaining graph was considered as unweighted. A vacant-surrounding-volume (V, FIG. 3) was then defined as: the number of clusters with distance at most S-1 from c; wherein c is a cluster and S is the distance in the graph between c and the closest cluster that contains a known structure. If a connected-component includes no occupied clusters, its vacant-surrounding-volume is undefined.

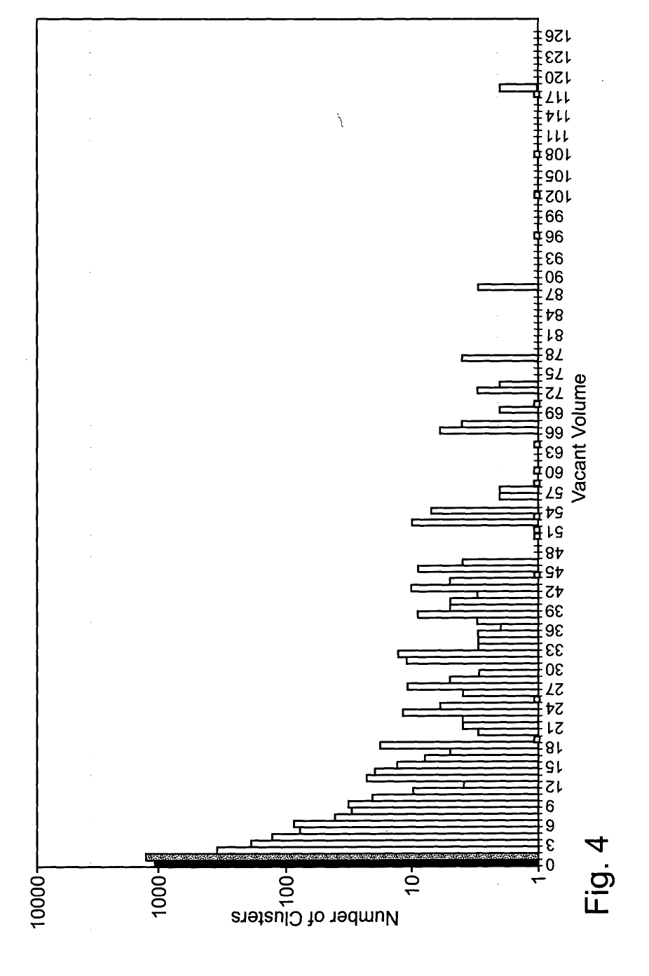

The distribution of vacant-surrounding-volumes over the clusters is shown in FIG. 4. In a previous study (Portugaly and Linial, 2000) it was shown that the vacant-surrounding-volume of a cluster is more informative than the number of steps (FIG. 3) with regards to the probability that the proteins it contains belong to a new fold.

Ordering Proteins According to a Probability to Belong to a New Superfamily

It was assumed that if protein A belongs to a cluster with higher vacant-surrounding-volume than protein B, then A is more likely to belong to a new superfamily. However, the probability that a protein belongs to a new superfamily if it is in a cluster with undefined vacant-surrounding-volume cannot be determined.

A list of clusters generated from 48,000 proteins, sorted by their vacant-surrounding-volume from large to small, and pointing to the proteins they contain, is available at http://www.cs.huji.ac.il/˜elonp/Superfamily-Targets. The assumption is that the higher ranked the protein, the better chance there is that it belongs to a new superfamily. Thus, the top members of this list contain candidates for Structural Genomics projects as obtained by the teachings of the present invention.

Validation Tests

Self-Validations

The present prediction method was validated by comparing major versions of SCOP (1.37, 1.41, 1.48, and 1.50). The prediction procedure was employed for each of the SCOP releases separately. Each of these runs was validated independently using ‘novel’ structures derived from the more recent releases of SCOP. The earlier release was designated the base release, while the later release was designated as the test release. It should be noted that the number of records has double from SCOP 1.37 to SCOP 1.50 and therefore such validation tests can be expected to be statistically sound.

Protein contained in the base SCOP release were ordered using only the structures from that release. Clusters were marked as occupied only if they contained proteins that were solved in the base SCOP release, while vacant-surrounding-volumes were assigned to clusters only with respect to these occupied clusters. The sequence test set used included sequences of the proteins that were not solved in the base SCOP release, but were solved in the test SCOP release. The structure test set was the superfamilies that did not appear in the base SCOP release but do appear in the test release.

Removing Redundancy of the Samples, and Marking Samples as New or Old

ProtoMap clusters of most fine level classification (level 1e-100) consist of almost identical proteins (pair similarity >1e-100). Sequence redundancy was removed from the test set by considering all proteins of a given level 1e-100 cluster as one sample. Because ProtoMap reflects hierarchical classification, it can be said that the sample belongs to a top-level cluster which represents all of the identical proteins. SCOP families that include any one of the proteins are considered to include this one sample. Since the proteins in the level 1e-100 cluster are almost identical, it can safely be assumed that if one protein in the 1e-100 cluster belongs to a family according to SCOP mapping generated herein, than all of the proteins in the cluster belong to that family. Each sample was marked with a plus if it belonged to a superfamily in the structure test set (i.e. it belongs to a new superfamily), and with a minus otherwise.

Ordering the Samples

The samples were ordered according to the vacant-surrounding-volume of the top-level cluster they belonged to. Samples that belonged to clusters with undefined vacant-surrounding-volume were not considered. N order to instate a strict ordering of the samples, a random order over all the samples that belonged to clusters with identical vacant-surrounding-volumes was defined.

Scoring the Order

Thus, a set of k pluses and l minuses that symbolized new and known superfamilies in the test sample, respectively was generated. A perfect prediction would have sorted all the pluses before the minuses. A scoring function over the set of orders of k pluses and l minuses was employed in order to score the order according to its distance from the perfect order. The score of each order was the number of position flips of pairs of adjacent pluses and minuses needed to transform the order to the perfect order; this score was exactly equal to summing over the positions of the pluses in the order (the first position indexed as 0). The final stage of the test was to find the P-value of the score, i.e., the probability that an order drawn from a uniform distribution of orders of k pluses and l minuses would gain a score such as this or higher).

Due to the random nature of the process, it was repeated 200 times for each pair. For all pairs of base and test releases, except for SCOP 1.48-SCOP 1.50, a total of 12 runs out of 1000 produced P-values above 1e-4. Half the runs for the tests SCOP 1.48-SCOP 1.50, produced P-values below 1e-3 and 90% produced P-values below 1 e-2.

Removing the Obvious Samples from the Test Set

The P-values for the ordering of all samples were very low (see above). However, one might argue, that the order only separates the obvious known superfamily samples from the rest of the samples. Thus, it could be argued that the only informative characteristic with regards to the probability of a sample to belong to new superfamilies, is whether or not it belongs to occupied clusters. In such a case, the order defined between two samples that belonged to clusters of vacant-surrounding-volume greater than 0, was uninformative.

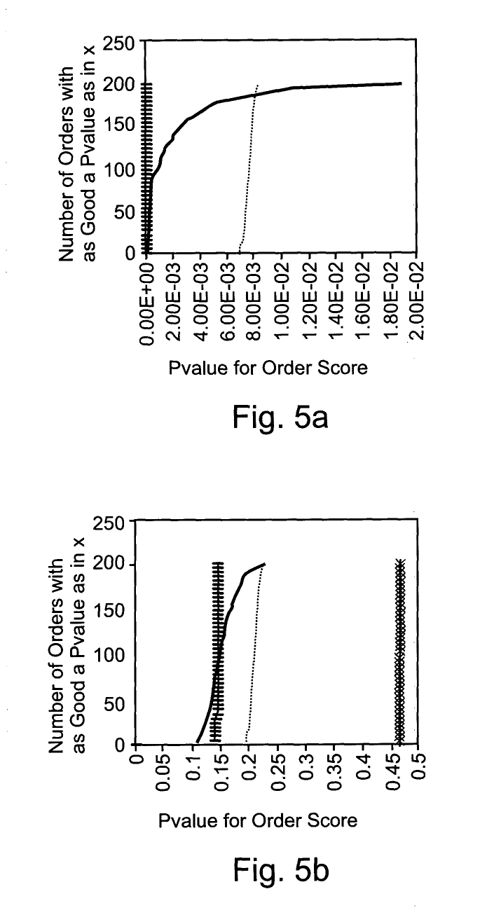

To verify that this was not the case, a subset of the samples was defined by removing samples that belonged to occupied clusters of the sequence test sets. This subset included “non-occupied” samples. The scores and P-values were recalculated for this subset in a manner similar to that described above. It was further tested that the order between two samples that belonged to clusters of vacant-surrounding-volume greater than 1 was informative. A subset of the non-occupied samples was defined by removing all samples that belonged to occupied neighboring clusters. This subset included “non-occupied-neighboring” samples. FIG. 5 illustrates the distribution of P-values for the 200 runs of the SCOP 1.37-SCOP 1.50 test for the two subsets.

Validation vs. PSI-BLAST Derived Predictions

The prediction performance of the present method was compared to predictions made using other methodologies. New orders were generated for the same six (base, test) sample sets on the basis of pair similarities according to the Smith-Waterman (SW) algorithm. Each protein of the sequence test set was tested as a SW query over Swissprot 36. Each protein tested was assigned the score of the best hit amongst the proteins that were solved according to the base SCOP release. Each sample (level 1e-100 cluster) in the test set was assigned the best score that any of the proteins composing the sample received. The samples were sorted according to their score, and the score and the P-value for the order was calculated as described. In order to evaluate the predictive capabilities of the present method versus state of the art methodologies, the above procedure was repeated using results from PSI-BLAST. PSI-BLAST is widely considered to be a very powerful tool for detection of remote homologues (Park et al., 1998) and for superfamily identification (Lindahl and Elofsson, 2000). Thus, the PSI-BLAST was tested against Swissprot 38, using the following parameters: Blosum62, gap penalty and gap extension-14 and 1; low complexity filter; iteration threshold E=0.001 and E-score threshold 100.

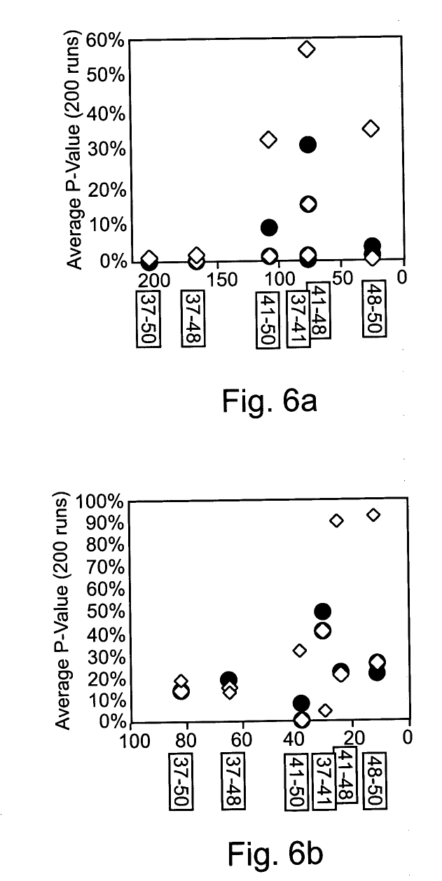

The Smith-Waterman derived orders performed poorly (see example in FIG. 5b). The average P-values for all 6 (base, test) sets, resultant from the present method, and PSI-BLAST are presented in FIGS. 6a-b.

As noted hereinabove, the present method initially relied upon a weak ordering approach (samples belonging to clusters with identical vacant-surrounding-volumes). For example, in the SCOP 1.37-SCOP 1.50 test, the 206 non-occupied samples were assigned only 18 vacancy-surrounding-volume values. This implies that information regarding the comparison of many pairs of proteins cannot be provided. In spite of this limitation, the orders produced by the present method performed well under the P-value test and in most cases better than PSI-BLAST derived orders.

To overcome the limitation described above, an order that was sorted by vacant-surrounding-volumes was defined, and PSI-BLAST was used to break ties between samples that had identical vacant-surrounding-volumes. As is illustrated in FIGS. 6a-b, the present method performed better than the PSI-BLAST derived orders, while the combined method typically performed better than both.

The SCOP 1.37-SCOP 1.50 and SCOP 1.37-SCOP 1.48 test sets were significantly larger, and as a result should probably be considered as being statistically more valid. As is evident from the results, using these test sets, the performance of the present method and the combined method is extremely good.

Test Conclusions

The results presented hereinabove clearly indicate that proteins that belong to occupied clusters are significantly (P-value ˜1e-5) less likely to belong to new superfamilies.

The prediction method of the present invention sorted proteins that had weak (non-occupied sample sets) or no (non-occupied-neighboring) pair sequence similarity to any solved protein. Although high resolution was not provided in all cases (proteins with identical vacant-surrounding-volumes), the “coarse sort” provided by the present method performed at least as well as a PSI-BLAST based sort. Furthermore, such a coarse sort can be refined using PSI-BLAST to provide results which are substantially better than either method alone.

Proposed List of Targets

The clusters at the top of the list generated by the present invention present possible targets for structural determination. All together 48,000 proteins were sorted; 6,000 proteins of the sorted proteins reside in 1274 clusters that were not occupied and were not neighboring occupied clusters and thus were ordered and placed at the top of the list. These proteins had a high probability of belonging to new superfamilies despite the lack of any significant pair sequence similarity to any known structure. As such, these proteins were further prioritized to select the best candidates for structural determination experiments. Such a list can be further filtered using information pertaining to the origin of the proteins in the phylogenetic tree, their size and their hydropathic nature. Among the 6096 proteins that are in non-occupied neighboring clusters, 1733 (included in 172 clusters) are in clusters of >5 proteins and are non-membranous. The list of the sorted proteins is available at http://www.cs.huji.ac.il/˜elonp/Targets. In addition, other filters including, but not limited to, host organism, size of the proteins in the clusters and others can also be used to further limit the target list.

### Example 2

Assigning Previously Uncharacterized Proteins with a “New Fold” Probability

This study was conducted in order to provide a computational-statistical method which can be used to assign previously uncharacterized proteins with a “new fold” probability. This approach employed two classifications of proteins, the sequence based classification of the protein space provided by ProtoMap (Yona, 1998) and the structure based classification provided by SCOP (Murzin, 1995). SCOP release 1.37 (5,741 natural protein entries that were registered at the PDB database prior to Oct. 20, 1997) was used. This release includes 11,748 records represented by 2,264 domains. The transformation from the number of PDB entries to the number of SCOP records and SCOP domains reflects: (i) parsing of proteins to their structural domains and (ii) grouping of entries in SCOP records that reflects the redundancy within PDB. The 2,264 domains are classified into 834 families, 593 super-families, 427 folds and 8 classes. Two more classes designed “proteins” and “non-protein” were not considered in this study.

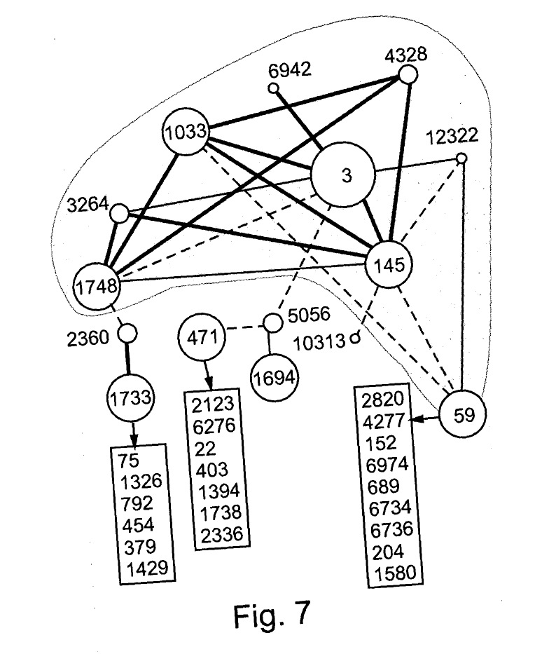

To construct the statistical model of this study, the most relaxed level of classification (level e=10-0) of ProtoMap version 2.0 was used. The 72,623 protein sequences of this level are classified into 13,354 clusters, 5,869 of which contain at least two proteins and 1,403 clusters have size 10 and above. Each cluster in ProtoMap has a weighted list of related clusters that form connected components of varying sizes and connectivities. In such a representation, weights (called quality) reflect relatedness among clusters. The lists of related clusters encode many biologically meaningful relations and form the basis for mapping the protein space as illustrated for the immunoglobulin superfamily (Yona, 1999), and the Ras superfamily (Linial, 1999 ). A ProtoMap graph, and the information that can be extracted from ProtoMap graphs are illustrated in FIG. 7 which illustrates a specific example of the globin family.

FIG. 7 illustrates a two dimensional presentation of all related clusters of cluster 3 (globulin family) and their immediate related clusters. Cluster 3 consists of 621 proteins representing myoglobins, globins and hemoglobins throughout the evolutionary tree. Inspection of the map of FIG. 7 indicates that additional globin-related clusters are linked to cluster 3 either directly or indirectly. For example, cluster 4328 contains proteins of C. Soyoae (deep-sea cold-seep clam) that are only weakly related to globins of other mollusca. Still, a connection can be traced between these globins and myoglobin of several mullusca and insecta (presented in cluster 145) and those of nematoda (cluster 1748). Another key feature of this graph is that a numerical value (quality) is assigned to pairs of related clusters to quantify their degree of proximity. Indeed, considering the level of proximity (described by the quality score) it is evident that edges connecting clusters of the globin family have higher score as compared to edges in the periphery.

Exploring the periphery of the globin family map (covered by the grey area) reveals numerous low score connections to cluster 59 (from clusters 145, 1,033 and 12,322). Cluster 59 is related to the globin family in that it contains flavohemoproteins (in combination with FAD-containing reductase domain). The other low score edges point to additional, non related local graphs (FIG. 7). This observation suggests that the graph of related clusters can be “cropped” at different thresholds, by eliminating all edges of significance below a given threshold. Each threshold yields a different scheme and thus, the protein universe is partitioned to connected components of different sizes and graph associations. Considering ProtoMap graph with all edges (referred to threshold 0.0) 37.7% of the clusters are within one connected component. Following cropping, at thresholds 0.1 and 0.3, the percentage of clusters within one connected component drops to 17.2% and 6.1%, respectively. Considering all 13,354 clusters in ProtoMap, the number of related clusters is on average 3.7 related clusters per cluster. However, the distribution of this value is very broad and is correlated with the cluster's size. For example, most singleton clusters (5,545 of 7,485) have no related clusters at all.

The Statistical Model

As described in Example 1, the present study postulates that proximity (i.e. small distances in the ProtoMap graph) is negatively correlated with similarity among protein features, including 3D structures. As such, clusters that are proximal in ProtoMap should tend to share a similar fold whereas clusters that are distant tend to have unrelated folds. This general hypothesis has been put to a number of biological tests that were manually evaluated. Such tests were carried out with respect to several biological features. In addition, several structurally based maps provided by FSSP were compared to ProtoMap clusters in order to evaluate the structural prediction capabilities of this methodology. In many instances, structurally related proteins that do not fall into the same ProtoMap cluster do, however, belong to neighboring clusters in ProtoMap graph (see Example 1 above).

The statistical model of this study of the present invention describes a cluster as “vacant” when it contains no known structures, and as occupied otherwise. A vacant cluster is said to be new when its (presently undetermined) corresponding fold is absent from SCOP, and old otherwise.

To construct this statistical model, the distribution of distances among occupied clusters in the ProtoMap graph was first determined in order to derive an estimate for two statistical distributions: (i) distances (within ProtoMap graph) from old clusters to occupied clusters and (ii) distances from new clusters to occupied clusters.

The first distribution is a good approximation for the typical distance distribution from a known structural fold to all clusters. The second distribution does the same for yet unsolved folds; the second distribution should be biased (as compared with the first distribution) towards larger distances. These distributions are the basis for evaluating the distances measured from all vacant clusters to occupied clusters.

Specifically, Baye's' rule is utilized to estimate the probability that a given cluster is new on the basis of these two distributions. This is effected using the measured distances from this cluster to all neighboring occupied clusters and the estimated number of folds in the protein space. Such probabilities were calculated for every vacant cluster in ProtoMap. The results were subsequently put to test by comparing predictions on all newly released protein structures as described hereinabove.

Estimating the probability of a vacant clusters to have new folds is based on three steps:

(i) relating each of the domains having a solved 3D structure (from the SCOP database) with its proper ProtoMap cluster;

(ii) determining a “representative fold” for each cluster based on the folds associated with all structural domains in that cluster;

(iii) determining distances within the ProtoMap graph from each representative fold to the neighboring folds, the distributions of these distances are used to create a statistical model for distances among those folds that are known and those that are yet to be discovered; and

(iv) Statistically estimating the probability that any protein has a new, yet undetermined fold, proteins that score the highest probability to represent a new fold constitute the list of preferred target proteins for structural determination.

Mapping SCOP Domains to Swissprot Protein Chains

The information from the PDB database is matched with that of the SwissProt records (hereinafter “SP-chain”) in order to accurately position the known structures against the ProtoMap graph; 2,264 representatives domains defined in SCOP (1.37) were used. Among these domains, 1,986 are successfully associated with SP-chains while the rest do not have a corresponding record in SwissProt database and as such cannot be matched.

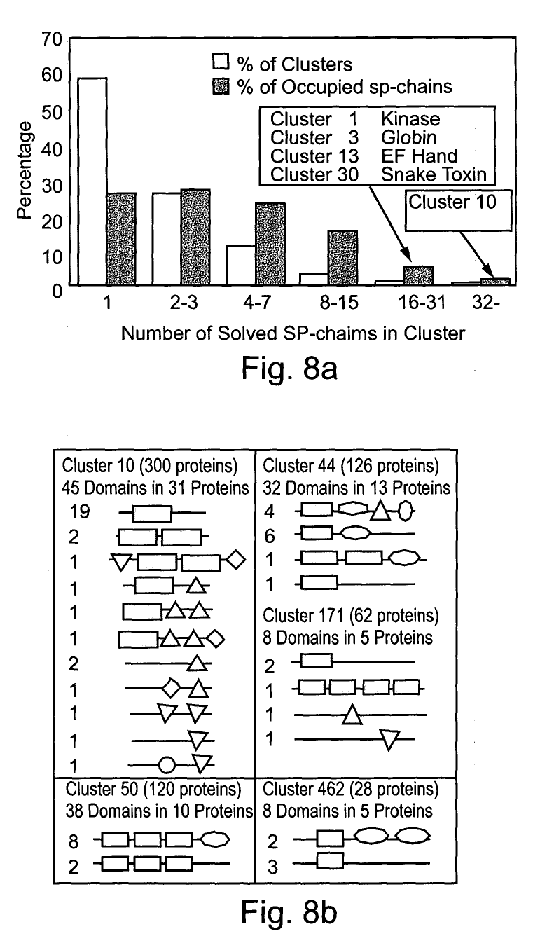

The association between structural domains and SP-chains is bidirectional. Of the 72,623 SP-chains, 1,688 are solved and as such belong to an occupied cluster. Of the 13,354 clusters in ProtoMap, 756 are occupied. The distribution of the number of solved SP-chains in each occupied cluster is shown in FIG. 8a. While 59% of the occupied clusters contain only one solved SP-chain (with one or more solved domain), 73% of the solved SP-chains are in clusters with two or more solved SP-chains. An occupied cluster is mapped to a specific fold if it contains an SP-chain that is mapped to that fold.

Assigning Representative Folds to ProtoMap Clusters

As is indicated by the mapping, there is no one-to-one correspondence between clusters and folds. Although it would be advantageous if a single representative fold is assigned to each ProtoMap cluster, it is not clear whether such a selection can be carried out. This is not an artifact of ProtoMap and SCOP. Many proteins are multi-domain, and an SP-chain may correspond to several domains, which usually have distinct folds. Thus, each occupied cluster is assigned the best representative fold according to the abundance of the fold in the cluster. For an occupied cluster with only a single domain, the representative fold is of course the fold of that domain. The same applies to those occupied clusters that have more than one domain, all the domains are mapped to the same fold. There are cases where all the domains in the cluster are positioned to different SP-chains (e.g., all 26 domains in clusters 3 and 10 domains in cluster 145 belong to a globin-like fold, FIG. 7) or to a single SP-chains. Examples of the latter case include the aspartate and ornithine carbamoyltransferases (cluster 101), LIM domains (cluster 123), Crystallins \overline{□} □ with ‘Greek key’ motif (cluster 132) and others. The rest of the occupied clusters have multiple folds that are mapped to the same cluster. Representative examples are illustrated in FIG. 8b.

Cluster 10 which includes the highest number of domains in a cluster (FIG. 8a) consists of 300 trypsin protease proteins. In this cluster, 45 domains are mapped to 31 solved SP-chains. These domains are associated with 5 different folds (FIG. 8b). Still, in 25 out of these 31 solved SP-chains a trypsin-like fold is represented. All other examples in FIG. 8b indicate clusters that contain several solved SP-chains, that are mostly multi-domain proteins.

Although a representative fold is assigned by selecting the fold that dominates most of the solved SP-chains in that cluster, in a small number of cases, where no dominant fold exists, the decision was made arbitrary (e.g., cluster 86 of the Lactate/malate dehydrogenase superfamily contains 30 SCOP domains that are associated with 15 occupied SP-chains, each having two different folds of the N- and C-terminal region). Of the 411 folds in SCOP 1.37 that are mapped to solved SP-chains, 329 folds were chosen as cluster representatives. Of the remaining 82 folds not chosen as representatives, most are coupled to a representative fold. Many of the folds not chosen as representatives are peptides (class 8 in SCOP) or very short domains that rarely dominate the cluster. Remarkably, only 80 solved SP-chains are not mapped to the representative fold of their cluster. These 80 SP-chains constitute less than 6.5% of the solved SP-chains in clusters that contain more than one solved SP-chain. This matching suggests that ProtoMap is selective for SCOP folds. That is, a cluster gathers proteins of the same fold, though not necessarily all proteins of that fold.

Predicting A Protein 's Probability to have a New Fold

The statistical estimates are based on an analysis of distances in the ProtoMap graph. As mentioned above, two distributions are determined: (i) distances from old clusters to occupied clusters and (ii) distances from new clusters to occupied clusters.

The computational procedures by which these two distributions are estimated are similar and are as follows:

(i) start from any occupied cluster;

(ii) consider all neighboring clusters, then their neighbors etc;

(ii) stop the scanning procedure when an occupied cluster is encountered. For the ‘old’ distribution (Dold), any occupied cluster terminates the procedure. In a ‘new’ distribution (Dnew), an occupied cluster halts the procedure only if its associated fold differs from the fold representing the cluster at which the scanning originated. Halting the scanning occurs due to an occupied cluster at a distance r from the origin cluster (this parameter will usually differ in the two scanning schemes).

The maximal vacant volume V is defined as the number of clusters whose distance from the origin is <r. If there are no occupied clusters in the connected component of the origin cluster then the maximal vacant volume V is defined as empty. The underlying assumption is that the size of the vacant volume is related to the probability that this cluster represents a new fold. Intuitively, one might expect that a cluster whose V is small will be represented by a known fold, because of its proximity to known structures, whereas a cluster whose V is large, corresponds to a new fold. This procedure is carried out separately with each of the 756 occupied clusters as origins. Based on the information collected from scanning ProtoMap graph the distributions of maximal vacant volumes are calculated to derive two conditional probability distributions: D[old|V] and D[new|V].

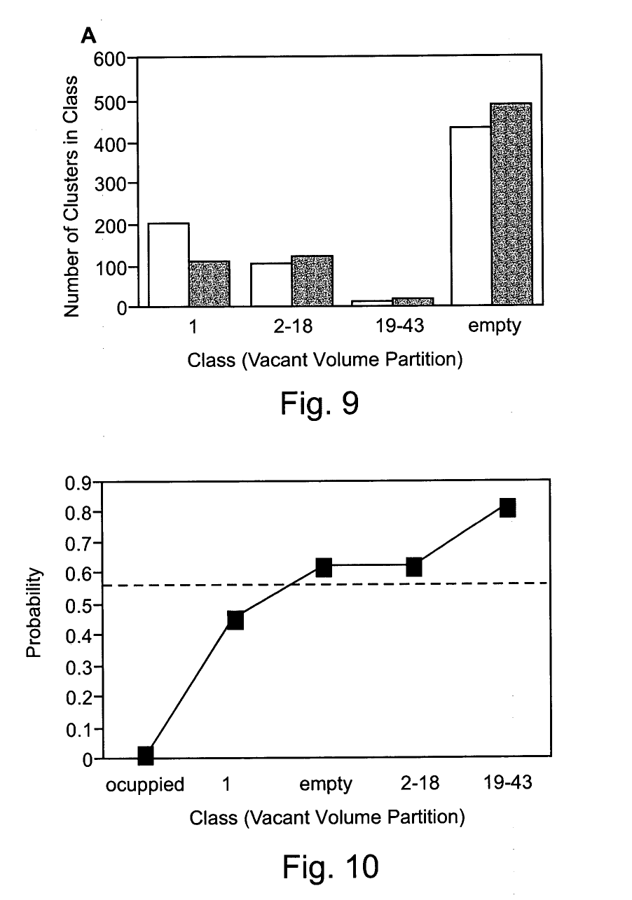

Evidently, V is strongly dependent on the properties of the ProtoMap graph. In order to extract maximum information from the ProtoMap graph, the scanning procedure is repeated for various graph-cropping thresholds. To optimize the partition of the empirical V values to discrete classes, Kullback-Leiber divergence (DKL) is used as a measure for the difference between the two distributions (Dold and Dnew). The DKL analysis was repeated in order to compare the two distributions at various thresholds (0.0, 0.1 and 0.3). The DKL value for threshold 0.1 is slightly higher than that for threshold 0.0. At threshold 0.3 the DKL values were lower than for the other thresholds and thus this information was not included in the prediction analysis. The distributions for D[old|V] and D[new|V] calculated from ProtoMap graph (threshold 0.0) and following clipping at threshold 0.1 are shown in FIG. 9. Note that in both cases, the two distributions (simulating old or new folds) are indeed biased as predicted by the model.

To determine the probability P[new|V] that a corresponding cluster will have a new fold given a cluster with a specific vacant volume V, one can apply conditional probability measure according to Baye's' rule:

$$P [ new \vert V ] = \frac{P [ V \vert new ] * P [ new ]}{D [ V ]} = \frac{{D}^{new} [ V ] * P [ new ]}{D [ V ]}$$

The prior probability of a new fold, P[new], is calculated based on the number of known folds (427, according to SCOP 1.37) while estimating the total number of folds, for which a rather conservative estimate of 1,000 is used (Chothia, 1992). Thus, P[new] can be considered as: 1−(total number of known folds)/(total number of folds) which equals in this case: 1−427/1000=0.573; while D[V], is given by the weighted sum over the two empirical distributions, as follows:

D[V]=Dnew[V]*P[new]+(1−P[new])*Dold[V]

FIG. 10 illustrates the values calculated for the probability that a cluster will have a new fold for various vacant volumes (at threshold 0.1). As evident therein, the probability function increases monotonically with the volume. This supports the initial hypothesis that distances in ProtoMap graphs reflect structural relatedness. Unfortunately, for many clusters which cannot be assigned a vacant volume (denoted empty) this analysis provides little information. For these clusters, the probability values are slightly above the a priori value (0.573).

Evaluation of the Predicted “New Fold” Probability

To evaluate this prediction, a test for evaluating membranous proteins was conducted. At present, few membranous proteins have been solved for structure (mostly classified in SCOP class 6). Therefore, clusters of membranous proteins are expected to have a high new fold probability. Over 1,000 clusters (representing about 20% of the SP-chains) with proteins having multiple membrane spanning regions were considered. The occurrence of these membranous clusters in the top probability classes is 6.5 fold higher than the overall occurrence of clusters in that class (see Table 1 below).

TABLE 1
Membranous clusters tests (combined)

| | | | | | | Ratio |
|---|---|---|---|---|---|---|
| classes | P(New\| | All clusters - | | Membranous | | Membranous/ |
| by V | V) | number (%) | | number (%) | | All clusters |
| Occupied | 0.00 | 756 | (5.7%) | 13 | (1.2%) | 0.2 |
| 1 | 0.46 | 1,123 | (8.4%) | 45 | (4.3%) | 0.5 |
| Empty | 0.62 | 9,651 | (72.3%) | 639 | (61.0%) | 0.8 |
| 2-18 | 0.63 | 1,111 | (8.3%) | 91 | (8.7%) | 1.0 |
| Add-0.0 | 0.76 | 405 | (3.0%) | 104 | (9.9%) | 3.3 |
| >19 | 0.82 | 308 | (2.3%) | 156 | (14.9%) | 6.5 |
| Total | | 13,354 | (100%) | 1,048 | (100%) | |

While membranous folds were strongly under represented in the training set and a clear preference towards high probability of being new was achieved, this data should not be considered representative of the number of membranous folds to be discovered. This test confirms that the probability function indeed assigns higher probabilities to membranous clusters as hypothesized. Table 1 shows that this tendency is kept steady throughout the probability range. Probabilities of the highest classes in thresholds 0.1 and 0.0 are in full agreement with the prediction. Consequently, both probabilities were combined to a final probability function by considering a cluster that is assigned to the highest class in threshold 0.0 while not being assigned to the highest class in threshold 0.1 (marked as Add 0.0*). Otherwise a cluster is assigned the probability of its class in threshold 0.1.

A more stringent evaluation is based on structural data not available during the statistical analysis. While the original analysis was performed using SCOP 1.37 (about 13,000 domains), re-evaluation was performed against SCOP 1.39 (about 18,000 domains). The process of associating domains with SP-chains using the records of SCOP 1.39 was repeated and 2,092 domains which constitute 404 folds were successfully mapped. The test included the group of SCOP 1.39 occupied clusters. Each of these clusters was assigned a fold based on the occupied SP-chains included therein. Due to ProtoMap's selectivity for folds, a 1.39 occupied cluster is most likely new if its (1.39) representative fold is new. This procedure obtained 388 new domains and 48 new folds. When considering all of the 1.39 occupied clusters, it was determined that 13.2% is the average percentile of new folds within all of these clusters.

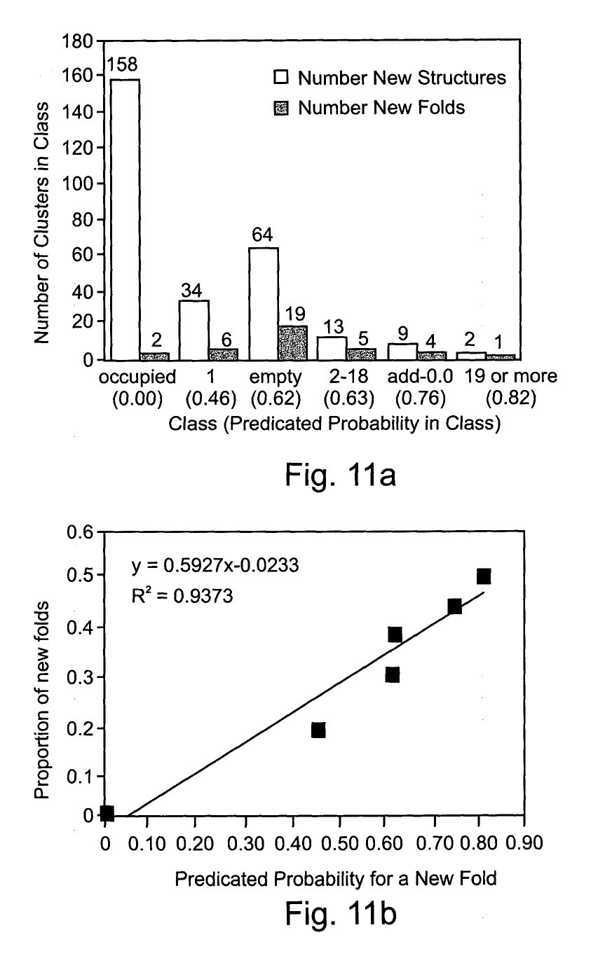

Given the vacant volume of these clusters, it is possible to test how well the predictions match the new assignments. Since clusters with high vacant volume have high probability to adopt a new fold, it is expected that a large fraction of these clusters are represented by new folds, i.e., the proportion of new clusters out of all clusters with the same vacant volume would increase as the vacant volume increases. The results are summarized in FIGS. 11a-b which illustrate a strong correlation between the predicted probability of being new and the proportion of new folds among the recently released structures. Hence the evaluation tests suggest that selecting targets from the top probability list will substantially accelerate the pace of new fold discovery.

List of Selected Targets for Structural Genomics

The list of targets exhibiting top probability scores contains 713 clusters (5.3% of all clusters), which account for 8.2% of the SP-chains. Following subtraction of clusters of membrane proteins and considering clusters that have more than 5 SP-chains, the number or clusters in this target list is reduced to 125. The list is further reduced to 94 clusters following updating against the PDB (dated up to November 1999). The complete list of target proteins is available at (http://www.cs.huji.ac.il/elonp/Target).

Out of this list 80 proteins were selected for further studies and are at different stages of expression, purification, crystallization and data collection (unpublished data).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent. application or sequence identified by their accession number was specifically and individually indicated to be incorporated herein by reference. In addition, citation or identification of any reference in this application shall not be construed as an admission that such reference is available as prior art to the present invention.

### REFERENCE CITED

Altschul, S. F., Gish, W., Miller, W., Myers, E. W., and Lipman, D. J. (1990). Basic local alignment search tool. J. Mol. Biol., 215, 403-410.

Altschul, S. F., and Koonin, E. V. (1998). Iterated profile searches with PSI-BLAST—a tool for discovery in protein databases. Trends Biochem. Sci., 23, 444-447.

Aravind, L., and Koonin, E. V. (1999). Gleaning non-trivial structural, functional and evolutionary information about proteins by iterative database searches. J. Mol. Biol., 287, 1023-1040.

Holm, L., and Sander, C. (1997). New structure-novel fold? Structure, 5, 165-171.

Kim, S. H. (1998). Shining a light on structural genomics. Nat. Struct. Biol., 5, 643-645.

Koehl, P., and Levitt, M. (1999). A brighter future for protein structure prediction. Nat. Struct. Biol., 6, 108-111.

Koonin, E. V., Tatusov, R. L., and Galperin, M. Y. (1998). Beyond complete genomes: from sequence to structure and function. Curr. Opin. Struct. Biol., 8, 355-363.

Lindahl, E., and Elofsson, A. (2000). Identification of related proteins on family, superfamily and fold level. J. Mol. Biol., 295, 613-625.

Linial, M., and Yona, G. (2000). Methodologies for target selection in structural genomics. Prog. Biophys. Mol. Biol., 73, 297-320

Marti-Renom, M. A., Stuart, A. C., Fiser, A., Sanchez, R., Melo, F., and Sali, A. (2000). Comparative protein structure modeling of genes and genomes [In Process Citation]. Annu. Rev. Biophys. Biomol. Struct., 29, 291-325.

Montelione, G. T., and Anderson, S. (1999). Structural genomics: keystone for a Human Proteome Project. Nat. Struct. Biol., 6, 11-12.

Moult, J., Hubbard, T., Fidelis, K., and Pedersen, J. T. (1999). Critical assessment of methods of protein structure prediction (CASP): round III. Proteins Suppi, 3, 2-6.

Murzin, A. G. (1996). Structural classification of proteins: new superfamilies. Curr. Opin. Struct. Biol., 6, 386-394.

Murzin, A. G., Brenner, S. E., Hubbard, T., and Chothia, C. (1995). SCOP: a structural classification of proteins database for the investigation of sequences and structures. J. Mol. Biol., 247, 536-540.

Neuwald, A. F., and Landsman, D. (1997). GCN5-related histone N-acetyltransferases belong to a diverse superfamily that includes the yeast SPT10 protein. Trends Biochem. Sci., 22, 154-155.

Olszewski, K. A., Yan, L., Edwards, D., and Yeh, T. (2000). From fold recognition to homology modeling: an analysis of protein modeling challenges at different levels of prediction complexity. Comput. Chem., 24, 499-510.

Park, J., Karplus, K., Barrett, C., Hughey, R., Haussler, D., Hubbard, T., and Chothia, C. (1998). Sequence comparisons using multiple sequences detect three times as many remote homologues as pairwise methods. J. Mol. Biol., 284, 1201-1210.

Portugaly, E., and Linial, M. (2000). Estimating the probability for a protein to have a new fold: A statistical computational model. Proc. Natl. Acad. Sci. USA, 97, 5161-5166.

Sali, A. (1998). 100,000 protein structures for the biologist. Nat. Struct. Biol., 5, 1029-1032.

Sippl, M. J., Lackner, P., Domingues, F. S., and Koppensteiner, W. A. (1999). An attempt to analyse progress in fold recognition from CASP1 to CASP3. Proteins, 37, 226-230.

Smith, T. F., and Waterman, M. S. (1981). Comparison of Biosequences. Adv. App. Math., 2, 482-489.

Terwilliger, T. C., Waldo, G., Peat, T. S., Newman, J. M., Chu, K., and Berendzen, J. (1998). Class-directed structure determination: foundation for a protein structure initiative. Protein Sci., 7, 1851-1856.

Wolf, Y. I., Grishin, N. V., and Koonin, E. V. (2000). Estimating the number of protein folds and families from complete genome data. J. Mol. Biol., 299, 897-905.

Yona, G., Linial, N., and Linial, M. (1999). ProtoMap—Automated classification of all proteins sequences: a hierarchy of protein families, and local maps of the protein space. Proteins, 37, 360-378.

Yona, G., Linial, N., and Linial, M. (2000). ProtoMap: automatic classification of protein sequences and hierarchy of protein families. Nucleic Acids Res., 28, 49-55.

Yona, G., Linial, N., Tishby, N., and Linial, M. (1998). A map of the protein space-an automatic hierarchical classification of all protein sequences. ISMB 6, 212-221.

What is claimed is:

1. A method of identifying a polypeptide candidate most likely to have undefined structural/functional elements comprising:
(a) classifying each cluster of a database of non-structurally clustered polypeptide clusters as:
(i) a first cluster, if it includes at least one structurally defined polypeptide; or
(ii) a second cluster, if it is devoid of structurally defined polypeptides;
(b) determining relational distances between at least one first cluster and a plurality of distinct second clusters according to at least one criteria; and
(c) identifying second clusters of said plurality of distinct second clusters which exhibit a relational distance from said at least one first cluster which is greater than a predetermined threshold, thereby identifying the polypeptide candidate most likely to have undefined structural/functional elements.

2. The method of claim 1, further comprising assigning each of said plurality of distinct second clusters a score according to a relational distance thereof from said at least one first cluster and at least one additional criteria selected from the group consisting of number of polypeptides in cluster, predominant host organism of cluster and average size of polypeptides in cluster.

3. The method of claim 1, wherein said database of non-structurally clustered polypeptide clusters is generated according to clustering of sequence data.

4. The method of claim 1, wherein said relational distances are determined according to sequence homology between polypeptides of said plurality of distinct second clusters and said at least one first cluster.

5. The method of claim 1, wherein each of said clusters of said database of non-structurally clustered polypeptide clusters is further classified according to the number of polypeptide constituents contained therein.

6. The method of claim 1, further comprising generating said database of non-structurally clustered polypeptide clusters according to clustering of sequence data prior to step (a).

7. The method of claim 6, wherein said classifying is repeated a predetermined number of times, each time for clusters generated according to a different homology threshold.

8. A method of determining putative structural/functional characteristics of a polypeptide having an unknown structure or function, the method comprising:
(a) classifying each cluster of a database of non-structurally clustered polypeptide clusters as:
(i) a first cluster, if it includes at least one structurally defined polypeptide; or
(ii) a second cluster, if it is devoid of structurally defined polypeptides;
(b) determining relational distances between at least one first cluster and a plurality of distinct second clusters according to at least one criteria;
(c) identifying second clusters of said plurality of distinct second clusters which exhibit a relational distance from said at least one first cluster which is less than a predetermined threshold; and
(d) determining the putative structural/functional characteristic of at least one polypeptide of said second clusters according to structural/functional characteristics of said at least one structurally defined polypeptide of said at least one first cluster, thereby determining putative structural/functional characteristics of a polypeptide having an unknown structure or function.

9. The method of claim 8, further comprising assigning each of said plurality of distinct second clusters a score according to a relational distance thereof from said at least one first cluster and at least one additional criteria selected from the group consisting of number of polypeptides in cluster, predominant host organism of cluster and average size of polypeptides in cluster.

10. The method of claim 8, wherein said database of non-structurally clustered polypeptide clusters is generated according to clustering of sequence data.

11. The method of claim 8, wherein said relational distances are determined according to sequence homology between polypeptides of said plurality of distinct second clusters and said at least one first cluster.

12. The method of claim 8, wherein each of said clusters of said database of non-structurally clustered polypeptide clusters is further classified according to the number of polypeptide constituents contained therein.

13. The method of claim 8, further comprising generating said database of non-structurally clustered polypeptide clusters according to clustering of sequence data prior to step (a).

14. The method of claim 13, wherein said classifying is repeated a predetermined number of times, each time for clusters generated according to a different homology threshold.

15. A system for identifying a polypeptide candidate most likely to have undefined structural/functional elements, the system comprising a processing unit being for executing a software application designed for:
(a) classifying each cluster of a database of non-structurally clustered polypeptide clusters as:
(i) a first cluster, if it includes at least one structurally defined polypeptide; or
(ii) a second cluster, if it is devoid of structurally defined polypeptides;
(b) determining relational distances between at least one first cluster and a plurality of distinct second clusters according to at least one criteria; and
(c) identifying second clusters of said plurality of distinct second clusters which exhibit a relational distance from said at least one first cluster which is greater than a predetermined threshold, thereby identifying the polypeptide candidate most likely to have undefined structural/functional elements.

16. The system of claim 15, wherein said software application is further designed for assigning each of said plurality of distinct second clusters a score according to a relational distance thereof from said at least one first cluster and at least one additional criteria selected from the group consisting of number of polypeptides in cluster, predominant host organism of cluster and average size of polypeptides in cluster.

17. The system of claim 15, wherein said relational distances are determined by said software application according to sequence homology between polypeptides of said plurality of distinct second clusters and said at least one first cluster.

18. The method of claim 15, wherein each of said clusters of said database of non-structurally clustered polypeptide clusters is further classified by said software application according to the number of polypeptide constituents contained therein.

19. The method of claim 15, wherein said software application is further designed for generating said database of non-structurally clustered polypeptide clusters according to clustering of sequence data.

20. A system for determining putative structural/functional characteristics of a polypeptide having an unknown structure or function, the system comprising a processing unit being for executing a software application designed for:
(a) classifying each cluster of a database of non-structurally clustered polypeptide clusters as:
(i) a first cluster, if it includes at least one structurally defined polypeptide; or
(ii) a second cluster, if it is devoid of structurally defined polypeptides;
(b) determining relational distances between at least one first cluster and a plurality of distinct second clusters according to at least one criteria;
(c) identifying second clusters of said plurality of distinct second clusters which exhibit a relational distance from said at least one first cluster which is less than a predetermined threshold; and
(d) determining the putative structural/functional characteristic of at least one polypeptide of said second clusters according to structural/functional characteristics of said at least one structurally defined polypeptide of said at least one first cluster, thereby determining putative structural/functional characteristics of a polypeptide having an unknown structure or function.

21. The system of claim 20, wherein said software application is further designed for assigning each of said plurality of distinct second clusters a score according to a relational distance thereof from said at least one first cluster and at least one additional criteria selected from the group consisting of number of polypeptides in cluster, predominant host organism of cluster and average size of polypeptides in cluster.

22. The system of claim 20, wherein said relational distances are determined by said software application according to sequence homology between polypeptides of said plurality of distinct second clusters and said at least one first cluster.

23. The method of claim 20, wherein each of said clusters of said database of non-structurally clustered polypeptide clusters is further classified by said software application according to the number of polypeptide constituents contained therein.

24. The method of claim 20, wherein said software application is further designed for generating said database of non-structurally clustered polypeptide clusters according to clustering of sequence data.
